(19) United States
(12) Patent Application Publication
(10) Pub. No.: US 2016/0113849 A1
(43) Pub. Date: Apr. 28, 2016

(54) FORMULATION FOR GRITTY FOAM DISPENSER

(71) Applicant: PIBED LIMITED, Denby (GB)

(72) Inventors: Louise GRIMADELL, Derby (GB); David Michael Ross CREAGHAN, Deerby (GB); John D. HINES, Audlem (GB); Pierre Bruno GRASCHA, Cormontreuil (FR)

(21) Appl. No.: 14/947,956

(22) Filed: Nov. 20, 2015

Related U.S. Application Data
(63) Continuation of application No. 13/804,209, filed on Mar. 14, 2013, now abandoned.
(60) Provisional application No. 61/679,377, filed on Aug. 3, 2012.

(51) Int. Cl. A61K 8/04; A61K 8/31; A61K 8/96; A61K 8/97; A61K 8/81; A61Q 19/10; A61K 8/92

(52) U.S. Cl. CPC A61K 8/046; A61Q 19/10; A61K 8/31; A61K 8/922; A61K 8/97; A61K 8/8111; A61K 8/965; A61K 2800/522; A61K 2800/48; A61K 2800/805; A61K 2800/654; A61K 2800/652; A61K 2800/651; A61K 2800/591

(57) ABSTRACT

Disclosed herein are foamable gritty foam compositions that can suspend particulate mechanical scrubbers with particles sized from about 100 microns to about 800 microns that is capable of being foamed through a non-aerosol, or unpressurized pump dispenser.

### CROSS REFERENCE TO RELATED U.S. PATENT APPLICATIONS

This patent application relates to U.S. utility patent application Ser. No. 61/679,377 filed on Aug. 3, 2012 entitled FORMULATION FOR GRITTY FOAM DISPENSER, filed in English, which is incorporated herein in its entirety by reference.

### FIELD

The present disclosure relates to formulations for a gritty foam dispenser.

### BACKGROUND

Currently, there are three (3) broad categories of hand cleansers that form the away from home skincare market. They are: lotion soaps, lotion soaps with suspended mechanical scrubbers and foam soaps. Lotion soaps are generally dispensed from soap dispenser pumps or pump top bottles. Lotion soaps with mechanical scrubbers are also dispensed through similar devices. Foaming soaps are dispensed from special pumps that mix air and liquid together through a porous media to produce foam.

Hand cleansers have been around for many years. The combination of ingredients in these types of soaps can be specified to achieve a broad range of cleaning characteristics. The viscosity of this form of soap is in the range of 5,000 to 30,000 cPoise and is generally tailored to be easily manipulated in the user's hand or pumped through a pump.

With the addition of mechanical scrubbers, the cleaning performance of lotion soaps can be enhanced. Lotion soaps with suspended mechanical scrubbers tend to have a viscosity range at the low end that is higher than that of basic lotion soaps. In order to provide the structure to suspend mechanical scrubbers of an appropriate size to provide cleaning benefits, these formulations must also have a critical strain force (or yield value) superior or equal to 30 dynes/cm² associated or not to non-Newtonian rheological behaviour, and more particularly plastic or thixotropic properties (pseudoplastic fluids being excluded from this requirement). The rheology of this form of soap must be viscoplastic (Casson, or Bingham, or dilatant, or thixotroplc, or rheopectic fluids) with a critical strain force which is preferably greater than or equal to 30 dynes/cm². The critical strain force is defined as that stress which must be applied before flow will start and, although related to viscosity, it is more dependent on the characteristics of the rheological additive used. The critical strain force has traditionally been determined by measuring the viscosity of the material at two speeds using a Brookfield Viscometer. As the speed of rotation of the spindle is increased materials that undergo shear thinning give a lower viscosity measurement.

The soap's critical strain force or the required yield value may be calculated as follows:

Yield Value (in dynes/cm²)*(V_{0.5rpm}−V₁ᵣₚₘ)/100

Required Yield Value (in dynes/cm²)=[4/3R(D−Do)g]
Where:
   R=particle radius (cm)
   D=particle density (g/cm³) Do=medium density (g/cm³)
   g=acceleration due to gravity=980 cm/s²

Foaming hand cleansers offer many benefits over lotion hand cleansers. Foam products are quicker and easier to use since they are delivered into the user's hand in a lathered format. Foam hand cleansers spread more effectively than lotion hand cleansers, providing better cleaning performance with less product. Since foaming hand cleansers are already lathered, there is less water needed in the cleaning process and the user can clean their hands in less time. The viscosity range of this form of hand cleanser is less than 100 cPoise and is tailored to be easily mixed with air through a porous media to produce foam from a pump.

The characteristics of foam soaps are such that they are not capable of suspending mechanical scrubbers of sufficient size to provide cleaning benefits. Current foam pump technologies mix air and liquid together and pass them through a porous media to make the foam. The porous media is generally-very fine and would slave out mechanical scrubbers.

Recently, a new type of pump capable of dispensing hand cleansers with mechanical scrubber in a foam format through a non-aerosol dispensing system has been developed (see U.S. Pat. No. 8,002,151). This has allowed for the creation of a new hand cleanser category. This category is foaming hand cleansers with mechanical scrubbers. This new category will allow the leveraging of all the benefits associated with foaming hand cleansers with the enhanced cleaning performance provided by mechanical scrubbers. Nevertheless, the requirement remains for a suitable hand cleanser.

Thus, it would be very beneficial if a hand cleanser could be formulated that can suspend particulate mechanical scrubbers with particles sized from about 100 microns to about 800 microns that decreases in viscosity when exposed to high shear rates and is capable of being foamed through a non-aerosol, or unpressurized pump dispenser.

If the formulation is too thin, the mechanical scrubbers will fall out of suspension. If the product is too thick, the amount of force required to foam the formulation become too high resulting in excessive operating force for the dispenser user and poor quality foam.

Prior art does not capture the requirement of the formulation to generate foam when mixed and dispensed with air from a non-aerosol dispenser. The assumption from the prior art is since it is soap, it will naturally foam. While this may be true if mechanical manipulation is done (rubbing in the hand), if the formulation does not have the desired characteristics, it will not create an acceptable foam through a non aerosol pump.

### SUMMARY

Provided herein are formulations for a gritty foam dispenser. An embodiment of a foamable gritty composition comprises constituents including a solvent present in a range from about 0.5% w/w to about 30.0% w/w, in which the solvent includes any one or combination of D'limonene and sunflower oil methyl ester. The formulations include a particulate scrubbing agent present in a range from about 1.0% w/w to about 8% w/w. Included is a surfactant present in a range from about 0.5% w/w to about 30.0% w/w, skin conditioner present in a range from 0.01% w/w to about 5.00% w/w, a non-Newtonian thickening agent present in a range from about 0.05% w/w to about 10% w/w, and water. The non-Newtonian thickening agent is selected to give the foamable gritty formulation a critical strain force greater than or equal to about 30 dynes/cm², and a viscosity in a range from about 500 cPoise to about 4000 cPoise such that the foamable gritty composition is dispensible as a foam from a non-aerosol foam dispenser.

The present disclosure also provides a method of producing and dispensing a gritty foam, the method comprising;

dispensing a foamable gritty composition from an unpressurized container having a dispenser pump configured to mix air with the foamable gritty composition under low pressure conditions during dispensing to form a gritty foam, the foamable gritty composition comprising

a) constituents including

a solvent present in a range from about 0.5% w/w to about 30.0% w/w, said solvent including any one or combination of D'limonene and sunflower oil methyl ester;

a particulate scrubbing agent present in a range from about 1.0% w/w to about 8% w/w;

a surfactant present in a range from about 0.5% w/w to about 30.0% w/w;

skin conditioner present in a range from 0.01% W/w to about 5.00% w/w; and

a non-Newtonian thickening agent present in a range from about 0.05% w/w to about 10% w/w;

water; and

b) wherein the non-Newtonian thickening agent is selected to give the foamable gritty composition a critical strain force greater than or equal to about 30 dynes/cm², and a viscosity in a range from about 500 cPoise to about 4000 cPoise such that said foamable gritty composition is dispensible as a foam from a an unpressurized foam dispenser.

A further understanding of the functional and advantageous aspects of the disclosure can be realized by reference to the following detailed description and drawings.

### BRIEF DESCRIPTION OF THE TABLES

Embodiments will now be described, by way of example only, with reference to the drawings, in which:

Table 1 shows a broad class of constituents that can be used to produce formulations for a gritty foam dispenser in accordance with the present invention;

Table 2 shows an exemplary formulation.

### DETAILED DESCRIPTION OF THE INVENTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

### DEFINITIONS

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

As used herein, the term “exemplary” or “example” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

As used herein, the phrase “non-Newtonian thickener” or “non-Newtonian thickening agent” means a category of fluids whose viscosity (resistance to deformation or shear forces) is influenced by the shear stress and/or by the time this force is applied.

As used herein, the phrase “critical strain force” (also referred to as “yield value”) refers to a term used in rheology in which the ‘critical strain force’ is the minimum force that must be applied to a material to induce viscous flow. The critical strain force is a measurable quantity (expressed in dynes/cm²). More particularly, both phrases are referring to the same phenomenon which is that up to a certain force exerted (the critical strain force), a visco-elastic material will exhibit “solid like” or “elastic” properties, i.e. it will not have a net flow. Above the critical strain force (or above the “yield value”) the same material will exhibit viscous properties, i.e. it will flow.

As an example, for beads (particulate scrubber particles in the present context) suspended in a fluid, as long as the strain force imparted by the suspended beads is less than the critical strain force (or less than the yield value), the beads will stay forever in suspension. If they exert a force higher than the critical strain (or yield value), then they will migrate over time, either floating to the top of the fluid or settling to the bottom.

As used herein, the phrase “non-aerosol dispenser” or “unpressurized dispenser” means a dispenser that does not have a stored propellant as the source of gas to create the foam structure within the hand cleanser.

“Foam” as used herein means a liquid and a gas mixed to form a mass of small bubbles that has a structure that lasts for a variable length of time.

The phrase “low pressure” in the context of producing a foam as used herein means a pressure of around an atmosphere or less such as when dispensing the foam from an unpressurized container. Typically when foams are dispensed from aerosol containers the foam is considered to be being dispensed under high “pressure” conditions.

The present disclosure provides a skin cleanser, which is a foamable gritty composition that can suspend mechanical scrubber particles having a size in the range from about 100 to about 800 microns that decreases in viscosity when exposed to high shear rates and is capable of being foamed through a non-aerosol (unpressurized) pump.

If the formulation is too thin (viscosity too low) and has a Newtonian rheological behaviour, the mechanical scrubbers will fall out of suspension. If the product is too thick (too viscous), the amount of force required to foam the formulation becomes too high resulting in excessive operating force for the dispenser user and a poor quality foam results.

While it is assumed in the field that if surfactants are present, it will naturally foam. However while this is a necessary condition, the inventors have found that it is not a sufficient condition to obtain a foam when particulate scrubbing agents are present. First, the fluid must be capable of becoming aerated and second it must be capable of stabilising the entrained air bubbles divided by fluid films. The presence of surfactants in a suitable concentration broadly satisfies the second of these requirements by providing a means of stabilising fluid films by setting up a surface tension gradient that acts to oppose draining forces and hence maintains the thickness of the films, avoiding collapse.

However, the presence of surfactants alone does not meet the first requirement, which requires that the fluid is sufficiently low in viscosity to allow for mixing with air under the conditions provided by the pump (i.e. without the requirement for excessive force). If the fluid is too viscous, air will not mix and hence bubbles will not form (and then cannot be stabilised as foam). This issue is central to providing a fluid of sufficiently low viscosity to allow for efficient mixing and hence foam formation, and also to contribute to suspending particles in the fluid when at rest.

### Constituents

### Solvents

In addition, the solvent can include aqueous (water) and non-aqueous components. The non-aqueous solvent is one or both of D-limonene and sunflower oil methyl ester. Other optional solvents that can be added include any one or combination of glycol ethers, esters, alcohols, terpenes other than D'Limonene, aromatic-free white spirit. A total amount of non-aqueous solvent present in the composition is in the range from about 0.5% w/w to about 30.0% w/w. The water is present in an amount to balance the total composition to 100% w/w weight.

### Scrubbing Agent

The particulate scrubbing agent is any one or combination of a vegetable based scrubbing agent, a synthetic based scrubbing agent and a mineral based scrubbing agent, and the particles have a size in a range from about 100 microns to about 800 microns, or in a range from about, or in a range from about 200 to about 700, or in a range from about 300 to about 500 microns. The particles may have any size in this range, or be in any narrow range than 100 to 800 microns, or there may be a mixture of any size of particles in this broad range present in the composition. The vegetable based scrubbing agent may be any one or combination of cornmeal, olive stone, walnut shells, ground fruit stones, ground corn meal, ground fruit shells. The synthetic based scrubbing agent may be any one or combination of polyethylene and polypropylene. The mineral based scrubbing agent may be any one or combination of ground shellfish, pumice, and silica. The particulate scrubbing agent present is present a range from about 1.0% w/w to about 8% w/w.

### Surfactants

The surfactant may include any one or combination of i) an anionic surfactant present in a range from about 1% w/w to about 20% w/w if present alone; ii) an amphoteric surfactant present in a range from about 0.5% w/w to about 5.0% w/w if present alone; iii) a non-ionic surfactant present in a range from about 0.5% w/w to about 20% w/w if present alone. If the surfactant is any combination of two or more of i), ii) or iii), the total amount of surfactant is also in the range from about 0.5% w/w to about 30.0%° w/w.

i) Amphoteric Surfactants

The amphoteric surfactant may be any one or combination of betaines, acyl ethylene diamines, amino-acids derivates, imidazolines. Alternatively, the amphoteric surfactant may be any one or combination of acylamphoacetate, acylamphodiacetate, acylamphodipropionate, sodium cocoglycinate, sodium alkyliminodipropionate, cocamidopropyl betaine, sodium cocoamphoacetate.

The betaine may be any one or combination of coco betaine and cocamidopropyl betaine.

ii) Non-Ionic Surfactants

The non-ionic surfactant may be any one or combination of glucosides, ethoxylated fatty alcohols, ethoxylated fatty acids, saccharose esters, sorbitan esters, alkanolamides, glycerol alkyl esters, polyoxyethylene glycol alkylphenol ethers,

iii) Anionic Surfactants

The anionic surfactant may be any one or combination of lauryl sulphates, lauryl ether sulphates, sulphosuccinates, carboxylates (i.e. sodium oleate), carboxylic acid esters (i.e. sodium dilaureth citrate), alkyl sulfate (i.e. sodium lauryl ether sulfate, ammonium alkyl sulfate, alkyl and alkyl-aryl sulfonates (i.e. sodium dodecyl benzene sulfonate), sulfosuccinates (i.e. disodium lauryl ether sulfosuccinate), isethionates (i.e. sodium cocoyl isethionate, ammonium cocoyl isethionate), taurates (i.e. sodium methyl cocoyl taurate, sodium methyl oleoyl taurate), acyl glutamates (i.e. sodium lauroyl glutamate, sodium cocoyl glutamate, disodium cocoyl glutamate), sarcosinate (i.e. cocoyl sarcosinate), alkylpolyglucosides (i.e. decyl glucoside, sodium lauryl glucose carboxylate, caprylyl/capryl glucoside).

### Skin Conditioner

The skin conditioner is any one or combination of a polyol, an anionic surfactant (examples: methyl gluceth-20, sodium laureth carboxylate, xylitylglucoside, sodium cocoyl glutamate, sodium cocoyl glycinate) a non-ionic surfactant (examples: PEG castor oil, PEG dimethicone, Glycereth cocoate), a cationic surfactant (examples: cocamidopropyl betainamide mea chloride, cocamidopropyl pg-dimonium chloride phosphate), an amphoteric surfactant (examples: lauramine oxide, undecylenamidopropyl betaine, sodium cocoamphoacetate), a cationic polymer, a quaternised gum, and a polyol.

The polyol may be any one or combination of glycerine and polyglycerin-6, propylene glycol, sorbitol, mannitol, erythritol, xylitol, arabitol, ribitol, dulcitol, lactitol, and maltitol.

The skin conditioner is present in a range from 0.01% w/w to about 5.00% w/w.

### Non-Newtonian Thickening Agents

The non-Newtonian thickening agent is selected to give the foamable gritty composition a critical strain force greater than or equal to about 30 dynes/cm², and a viscosity in a range from about 500 cPoise to about 4000 cPoise, and preferably about 1000 cPoise to about 3000 cPoise, and most preferably about 2000 cPoise to about 2500 cPoise such that the foamable gritty composition is dispensible as a foam from a non-aerosol foam dispenser.

The non-Newtonian thickening agent may be any one or combination of synthetic polymers and natural thickeners. The synthetic polymer may be selected from the group consisting of acrylate copolymers. The synthetic polymer may be any one or combination of acrylates/C10-30 alkyl acrylate crosspolymer and carbomers.

The natural thickener may be any one or combination of xanthan gum, guar gum, quaternised guar gum, alginate, bentonite and fumed silica.

The non-Newtonian thickening agent is present in a range from about 0.05% w/w to about 10% w/w.

### Antioxidants

Antioxidants may be included in the formulations which are included to limit the risk of oxidation of d-limonene (becomes a sensitizer when oxidised). The antioxidants may include, but are not limited to: butylhydroxytoluene (BHT), butylhydroxyanizole, 1,2-dihydroxybenzene, p-coumarine acid, caffeic acid, sodium sulfite, sodium metasulfite, ferrulic acid, tyrosol, quercetin, chlorogenic acid, oleuropein hydroxytyrosol, ascorbic acid, phenolic acid, propyl gallate, α-tocopherol, β-tocopherol, γ-tocopherol, δ-tocopherol, tetradibutyl-pentaerithrityl hydroxyhydrocinnamate. The antioxidants may be present in an amount ranging from about 0.01 to 1.0% w/w.

### Non-Aerosol, Unpressurized Pump

A useful non-aerosol, unpressurized pump that may be used is disclosed in U.S. Pat. No. 8,002,151, which is incorporated herein by reference in its entirety. The unpressurized container used for dispensing the present foamable gritty composition will generate pressure when the gritty composition and air are introduced into a mixing chamber by mechanical actuation of a dispensing feature(s), and this mechanical actuation can be initiated by a user actuating the dispensing feature(s) or by sensor activated dispensing feature(s) when the presence of a user is detected.

Using such an unpressurized pump, the present disclosure provides a method of making and dispensing a gritty foam. The method involves introducing a predetermined quantity of air under pressure to a first side of a microporous air sparging element located upstream from an outlet while introducing, concurrently with the introduction of the air, a predetermined amount of the foamable gritty composition under pressure to a foamable gritty composition inlet of a mixing chamber located on a second side of the microporous air sparging element and located upstream from the outlet, such that a ratio of air to foamable gritty composition has a greater proportion of air to foamable gritty composition. The method involves forcing the air through the microporous sparging element into the mixing chamber to form a plurality of bubbles and mixing the plurality of bubbles and the foamable gritty composition in the mixing chamber thereby creating a gritty foam and dispensing the gritty foam to a user in shots through the outlet from the mixing chamber.

The air may be introduced in a predetermined quantity and the foamable gritty composition may be introduced in a predetermined quantity

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

TABLE 1

| | In the range (% w/w) | | | |
|---|---|---|---|---|
| Formulation to | | | More | Specific |
| contain | Typically | Preferably | preferably | examples |
| Thickener/ | 0.05-10.0 | 1.0-2.0 | 1.5-1.7 | Acrylic thickeners |
| suspending | | | | such as acrylates |
| agent | | | | copolymer, |
| | | | | natural thickeners |
| | | | | such as xanthan |
| | | | | gum |
| Scrubbing | 1.0-8.0 | 4.0-8.0 | 6.5-7.5 | Vegetable based |
| agent | | | | scrubbing agents |
| | | | | such as |
| | | | | cornmeal, olive |
| | | | | stone, walnut |
| | | | | shell |
| Anionic | 5.5-11.5 | 8.0-10.0 | 8.0-10.0 | Lauryl sulphates, |
| surfactants | | | | lauryl ether |
| | | | | sulphates, |
| | | | | sulphosuccinates, |
| Amphoteric | 0.8-3.0 | 1.0-2.0 | 1.0-2.0 | Betaines such as |
| surfactants | | | | coco betaine |
| Nonionic | 0.7-3.0 | 1.0-2.0 | 1.0-2.0 | Coco-glucoside, |
| surfactants | | | | decyl glucoside |
| Skin | 0.01-5.0 | 2.0-4.0 | 3.0-4.0 | Polyglycerin-6, |
| conditioner | | | | glycerine |
| Solvent | 0.5-30.0 | 1.0-10.0 | 1.0-6.0 | D'Limonene, |
| | | | | sunflower oil |
| | | | | methyl ester |
| Water | 29.5-91.44 | 62.0-82.0 | 66.8-78.0 | |

TABLE 2

| | | | | Nominal | | Nominal | |
|---|---|---|---|---|---|---|---|
| | | | Nominal | Active | | Active | |
| CAS | INCI | Generic Name | Code | Level % | % w/w | Content % | Function |
| 7732-18-5 | Aqua | Water | 11 210 355T | 100.00 | 48.02999 | 75.23212 | Solvent/diluent |
| N/A | Acrylates Copolymer | Aqua SF-1 | 11 105 567T | 30.00 | 5.15000 | 1.54500 | Thickener/Suspending Agent |
| (Polymer) | | | | | | | |
| 52-51-7 | 2-Bromo-2-Nitropropane-1,3- | Euxyl K145 | 11 105 560T | 14.00 | 0.30000 | 0.04200 | Preservative |
| | Diol | | | | | | |
| 2-682-20-4 | Methylisothiazolinone | | | 0.12 | | 0.00036 | Preservative |
| 29-172-55-4 | Methylchloroisothiazolinone | | | 0.37 | | 0.00111 | Preservative |
| 10377-60-3 | Magnesium Nitrate | | | <1 | | <0.003 | Preservative Stabiliser |
| 7786-30-3 | Magnesium Chloride | | | <1 | | <0.003 | Preservative Stabiliser |
| 66071-96-3 | Corn (Zea Mays) Meal | Cornmeal (irradiated) | 11 105 620T | 100.00 | 7.50000 | 7.50000 | Scrubbing Agent |
| 144538-83-0 | Tetrasodium Iminodisuccinate | Sodium | 11 105 579T | 78.00 | 0.10000 | 0.07800 | Chelating Agent |
| | | Iminodisuccinate | | | | | |
| 9004-82-4 | Sodium Laureth Sulfate | SLES, Preserved | 11 200 968T | 27.50 | 18.00000 | 4.95000 | Anionic Surfactant |
| 139-96-8 | TEA-Lauryl Sulfate | TEA Lauryl Sulphate | 11 105 207T | 40.00 | 8.00000 | 3.20000 | Anionic Surfactant |
| 5989-27-5 | Limonene | D'Limonene | 11 125 253T | 99.90 | 1.00000 | 0.99900 | Solvent |
| 128-37-0 | BHT | | | 0.10 | | 0.00100 | Antioxidant |
| 61789-40-0 | Cocamidopropyl Betaine | Betaine Coco Base | 11 105 204T | 30.00 | 4.50000 | 1.35000 | Amphoteric Surfactant |
| 25618-55-7 | Polyglycerin-6 | Polyglycerin-6 | 11 105 631T | 79.00 | 4.00000 | 3.16000 | Skin Conditioner |
| 56-81-5 | Glycerin | | | 1.00 | | 0.04000 | Skin Conditioner |
| 1310-73-2 | Sodium Hydroxide | Sodium Hydroxide | 11 105 107T | 47.00 | 0.12000 | 0.05640 | Neutraliser |
| 141464-42-8 | Coco-Glucoside | Glucopon 650 EC | 11 105 484T | 51.50 | 3.00000 | 1.54500 | Nonionic Surfactant |
| 77-92-9 | Citric Acid | Citric Acid | 11 105 114T | 100.00 | 0.30000 | 0.30000 | pH Adjuster |
| 7647-14-5 | Sodium Chloride | Sodium Chloride | 11 105 161T | 100.00 | 0.00001 | 0.00001 | Sodium chloride could |
| | | | | | | | possibly be needed for batch |
| | | | | | | | correction |
| | | | | | | | (Viscosity Adjuster) |

| | | |
|---|---|---|
| Total | 100.00000 | 100.00000 |

Therefore what is claimed is:

1. A foamable gritty composition, comprising:
a) constituents including
a solvent present in a range from about 0.5% w/w to about 30.0% w/w, said solvent including any one or combination of D'limonene and sunflower oil methyl ester;
a particulate scrubbing agent present in a range from about 1.0% w/w to about 8% w/w;
a surfactant present in a range from about 0.5% w/w to about 30.0% w/w;
skin conditioner present in a range from 0.01% w/w to about 5.00% w/w; and
a non-Newtonian thickening agent present in a range from about 0.05% w/w to about 10% w/w;
water; and
b) wherein the non-Newtonian thickening agent is selected to give the foamable gritty formulation a critical strain force greater than or equal to about 30 dynes/cm², and a viscosity in a range from about 500 cPoise to about 4000 cPoise such that said foamable gritty composition is dispensible as a foam from a non-aerosol foam dispenser.

2. The composition according to claim 1 wherein the surfactant includes any one or combination of
i) an anionic surfactant present in a range from about 1% w/w to about 20% w/w if present alone;
ii) an amphoteric surfactant present in a range from about 0.5% w/w to about 5.0% w/w if present alone;
iii) a non-ionic surfactant present in a range from about 0.5% w/w to about 20% w/w if present alone; and
wherein a total amount of surfactant of any combination of surfactants present is in a range from about 0.5% w/w to about 30.0% w/w.

3. The composition according to claim 1 wherein the solvent further includes any one or combination of glycol ethers, esters, alcohols, terpenes other than D'Limonene, aromatic-free white spirit, and wherein a total amount of solvent present is in said range from about 0.5% w/w to about 30.0% w/w.

4. The composition according to any one of claims 1 wherein the particulate scrubbing agent is any one or combination of a vegetable based scrubbing agent, a synthetic based scrubbing agent and a mineral based scrubbing agent, and wherein the scrubbing agent has a size in a range from about 100 microns to about 800 microns.

5. The composition according to claim 4 wherein the vegetable based scrubbing agent is any one or combination of cornmeal, olive stone, walnut shells, ground fruit stones, ground corn meal, ground fruit shells.

6. The composition according to claim 4 wherein the synthetic based scrubbing agent is any one or combination of polyethylene and polypropylene.

7. The composition according to claim 4 wherein the mineral based scrubbing agent is any one or combination of ground shellfish, pumice, and silica.

8. The composition according to claim 2 wherein the anionic surfactant is selected from the group consisting of lauryl sulphates, lauryl ether sulphates, sulphosuccinates, carboxylates (i.e. sodium oleate), carboxylic acid esters (i.e. sodium dilaureth citrate), alkyl sulfate (i.e. sodium lauryl ether sulfate, ammonium alkyl sulfate, alkyl and alkyl-aryl sulfonates (i.e. sodium dodecyl benzene sulfonate), sulfosuccinates (i.e. disodium laurylether sulfosuccinate), isethionates (i.e. sodium cocoyl isethionate, ammonium cocoyl isethionate), taurates (i.e. sodium methyl cocoyl taurate, sodium methyl oleoyl taurate), acyl glutamates (i.e. sodium lauroyl glutamate, sodium cocoyl glutamate, disodium cocoyl glutamate), sarcosinate (i.e. cocoyl sarcosinate), alkylpolyglucosides (i.e. decyl glucoside, sodium lauryl glucose carboxylate, caprylyl/capryl glucoside).

9. The composition according to claim 2 wherein the amphoteric surfactant is any one or combination of betaines, acyl ethylene diamines, amino-acids derivates, imidazolines.

10. The composition according to claim 2 wherein the amphoteric surfactant is any one or combination of acylamphoacetate, acylamphodiacetate, acylamphodipropionate, sodium cocoglycinate, sodium alkyliminodipropionate, cocamidopropyl betaine, sodium cocoamphoacetate.

11. The composition according to claim 9 wherein said betaine is selected from the group consisting of coco betaine and cocamidopropyl betaine.

12. The composition according to claim 2 wherein the non-ionic surfactant is any one or combination of glucosides, ethoxylated fatty alcohols, ethoxylated fatty acids, saccharose esters, sorbitan esters, alkanolamides, glycerol alkyl esters and polyoxyethylene glycol alkylphenol ethers.

13. The composition according to claim 1 wherein said skin conditioner is any one or combination of a polyol, an anionic surfactant, a non-ionic surfactant, a cationic surfactant, an amphoteric surfactant, a cationic polymer, a quaternised gum, and a polyol.

14. The composition according to claim 13 wherein said polyol is any one or combination of glycerine and polyglycerin-6, propylene glycol, sorbitol, mannitol, erythritol, xylitol, arabitol, ribitol, dulcitol, lactitol, and maltitol.

15. The composition according to claim 1 wherein said thickening agent is any one or combination of synthetic polymers and natural thickeners.

16. The composition according to claim 15 wherein said synthetic polymer is selected from the group consisting of acrylate copolymers.

17. The composition according to claim 15 wherein said synthetic polymer is selected from the group consisting of acrylates/C10-30 alkyl acrylate crosspolymer, carbomers, and combinations thereof.

19. The composition according to claim 15 wherein said natural thickener is selected from the group consisting of xanthan gum, guar gum, quaternised guar gum, alginate, bentonite and fumed silica.

20. The composition according to claim 1 including an antioxidant present in a range from about 0.01 to about 1.0% w/w.

21. The composition according to claim 20 wherein said antioxidant is any one or combination of butylhydroxytoluene (BHT), butylhydroxyanizole, 1,2-dihydroxybenzene, p-coumarine acid, caffeic acid, sodium sulfite, sodium metasulfite, ferrulic acid, tyrosol, quercetin, chlorogenic acid, oleuropein hydroxytyrosol, ascorbic acid, phenolic acid, propyl gallate, α-tocopherol, β-tocopherol, γ-tocopherol, δ-tocopherol, and tetrad ibutyl-pentaerithrityl hydroxyhydrocinnamate.

22. A method of producing and dispensing a gritty foam, the method comprising;
dispensing a foamable gritty composition from an unpressurized container having a dispenser pump configured to mix air with the foamable gritty composition under low pressure conditions during dispensing to form a gritty foam, the foamable gritty composition comprising
a) constituents including
a solvent present in a range from about 0.5% w/w to about 30.0% w/w, said solvent including any one or combination of D'limonene and sunflower oil methyl ester;
a particulate scrubbing agent present in a range from about 1.0% w/w to about 8% w/w;
a surfactant present in a range from about 0.5% w/w to about 30.0% w/w;
skin conditioner present in a range from 0.01% w/w to about 5.00% w/w; and
a non-Newtonian thickening agent present in a range from about 0.05% w/w to about 10% w/w;
water; and
b) wherein the non-Newtonian thickening agent is selected to give the foamable gritty composition a critical strain force greater than or equal to about 30 dynes/cm², and a viscosity in a range from about 500 cPoise to about 4000 cPoise such that said foamable gritty composition is dispensible as a foam from a an unpressurized foam dispenser.

23. The method of claim 22 including
introducing a predetermined quantity of air under pressure to a first side of a microporous air sparging element located upstream from an outlet;
introducing, concurrently with the introduction of the air, a predetermined amount of said foamable gritty composition under pressure to a foamable gritty composition inlet of a mixing chamber located on a second side of the microporous air sparging element that is located upstream from said outlet, such that a ratio of air to foamable gritty composition has a greater proportion of air to foamable gritty composition;
forcing the air through the microporous sparging element into the mixing chamber to form a plurality of bubbles;
mixing the plurality of bubbles and the foamable gritty composition in the mixing chamber thereby creating a gritty foam; and
dispensing the gritty foam to a user in shots through said outlet from the mixing chamber.

24. A method as claimed in claim 23 wherein the air is introduced a predetermined quantity and the foamable gritty composition is introduced in a predetermined quantity.
